(19) United States
(12) Patent Application Publication
(10) Pub. No.: US 2011/0098617 A1
(43) Pub. Date: Apr. 28, 2011

(54) TORSO ASSIST OTHOSIS

(76) Inventors: Daniel L. Ferguson, Appleton, WI (US); Loweel Mick N. Batton, Neenah, WI (US)

(21) Appl. No.: 12/904,801

(22) Filed: Oct. 14, 2010

Related U.S. Application Data
(60) Provisional application No. 61/254,287, filed on Oct. 23, 2009.

(51) Int. Cl. A61F 5/00

(52) U.S. Cl. 602/16

(57) ABSTRACT

An orthopedic support device with an upper body engaging portion and a lower body engaging portion connected by a hinge or pivot and including a spring assist device. The orthopedic support device configured to provide assistance to a user in returning the user's torso from a forward leaned position to a vertical position. The device may be selectively engaged to permit the user to sit normally while wearing the device. The spring assist device may include a coil spring. The spring may be changeable to adapt to the level of assistance an individual user requires. The spring assist device may include a preload adjustment.

### CROSS-REFERENCE TO RELATED APPLICATION

The present application claims priority to U.S. Provisional Application Ser. No. 61/254,287, filed on Oct. 23, 2009, the disclosure of which is incorporated herein by reference.

This device is intended to assist patients with camptocormia but could be used for other conditions or illnesses which cause difficulty standing and walking erect. The device performs the following functions:
1. Holds the patient erect while standing or walking
2. Allows patient freedom to bend at the waist and then provides assistance to resuming an upright position.
3. When disengaged, allows patient to assume seated position while holding the torso erect.

The Torso Assist Othosis (TAO) of the present disclosure is similar to the Reciprocating Gait Othosis (RGO) in form and fit. Unlike the RGO, the TAO has no reciprocating mechanism, nor does it restrict dynamic axial extension of the spine. Instead, the hip joint may be fitted with a spring mechanism which is preferably sized to counterbalance the specific torso weight and muscle capability of each patient. This selection of spring characteristics allows the patient the freedom to bend at the waist and return to an upright standing position with minimal effort.

The TAO design of the present disclosure employs a torsion coil spring to provide the counterbalancing force but other types could be used. Examples are spiral-wound torsion springs and torsion bars. Other types of restorative devices could also be used. Examples are spring struts, spring loaded cam followers, or even motorized linear actuators.

Benefits that may be provided by the preferred torsion spring mechanism are:

1. Adjustable torsion spring strength based on weight or needs of patient

2. Adjustable spring pre-load

3. High-strength cam action plunger latching pin

4. Self latching feature

5. Precision roller bearing movement

In order to simplify use and implementation, the TAO was designed to be compatible with standard lightweight hip joint and spinal othosis components. This makes it possible to add the TAO mechanism to a standard RGO if both functions are needed by the patient. While the convention designs for similar othoses do not permit them to be used simultaneously, extensible links (either spring loaded or motorized) could be employed in a traditional RGO which would provide the additional degree of freedom afforded by the TAO. It is intended that such embodiments are within the scope of the present disclosure.

### BRIEF DESCRIPTION OF THE DRAWINGS

The accompanying drawing figures, which are incorporated in and constitute a part of the description, illustrate several aspects of the invention and together with the description, serve to explain the principles of the invention. A brief description of the figures is as follows:

FIG. 1 is a side view of a torso assist othosis (TAO) according to the present disclosure, with a user of the device shown in a torso upright position.

FIG. 2 is a side view of the TAO and user of FIG. 1, with the user shown in a forward-lean with the TAO supporting some of user's torso weight.

FIG. 3 is a side view of the TAO and user of FIG. 1, with the user shown in a sitting position and a spring assist of the TAO disengaged.

FIG. 4 is a front cross-sectional view of a right side pivot assembly for the TAO of FIG. 1.

FIG. 5 is a closer side view of an upper anchor block of the pivot assembly of FIG. 4.

FIG. 6 is a closer side view of a lower anchor block of the pivot assembly of FIG. 4.

FIG. 7 is a closer view of a central fastener of the pivot assembly of FIG. 4.

FIG. 8 is a side view of a main body of the pivot assembly of FIG. 4.

FIG. 9 is a front cross-sectional view of the main body of FIG. 8.

FIG. 10 is a side of the main body of FIG. 8 with a spring cover in place.

FIG. 11 is a side view of the spring cover of FIG. 10.

FIG. 12 is a front view of a pair of springs for use with the TAO of FIG. 1.

FIG. 13 is a side view of one of the springs of FIG. 12.

FIG. 14 is a side view of an alternative embodiment of a main body for use with the pivot assembly of FIG. 4.

FIG. 15 is a front cross-sectional view of the main body of FIG. 14.

FIG. 16 is a side view of a second alternative embodiment of a main body for use in a TAO according to the present disclosure.

FIG. 17 is a front cross-sectional view of the main body of FIG. 16.

### DETAILED DESCRIPTION

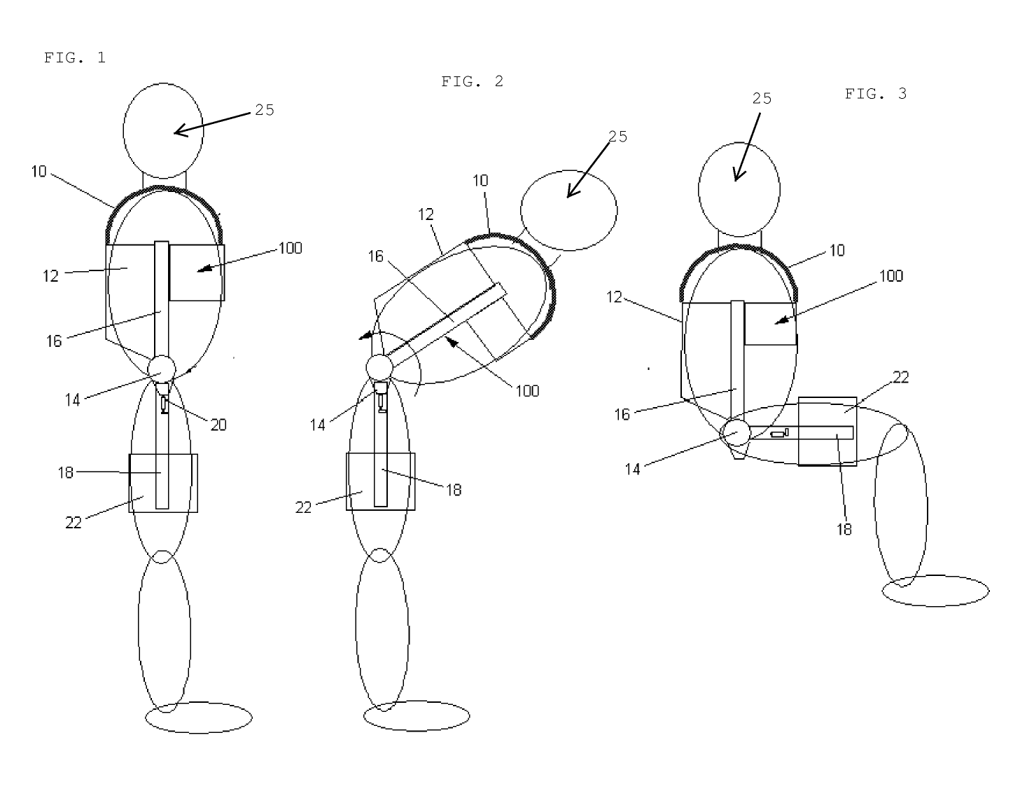

FIGS. 1 to 3 illustrate a preferred embodiment 100 of a TAO according to the present disclosure and will be referred to in the description as an illustrative and non-limiting example. Applicants anticipate that various options and alternatives will be apparent and it is not intended limit the present disclosure in any way. TAO 100 is illustrated as being worn or used by a user 25.

The main components of the TAO are as follows and illustrated in FIG. 1 of the drawings:

Shoulder harness 10

Pelvic girdle and frame 12 providing an upper body engaging portion

Hip joint pivot 14 with upper 16 and lower 18 attachment arms

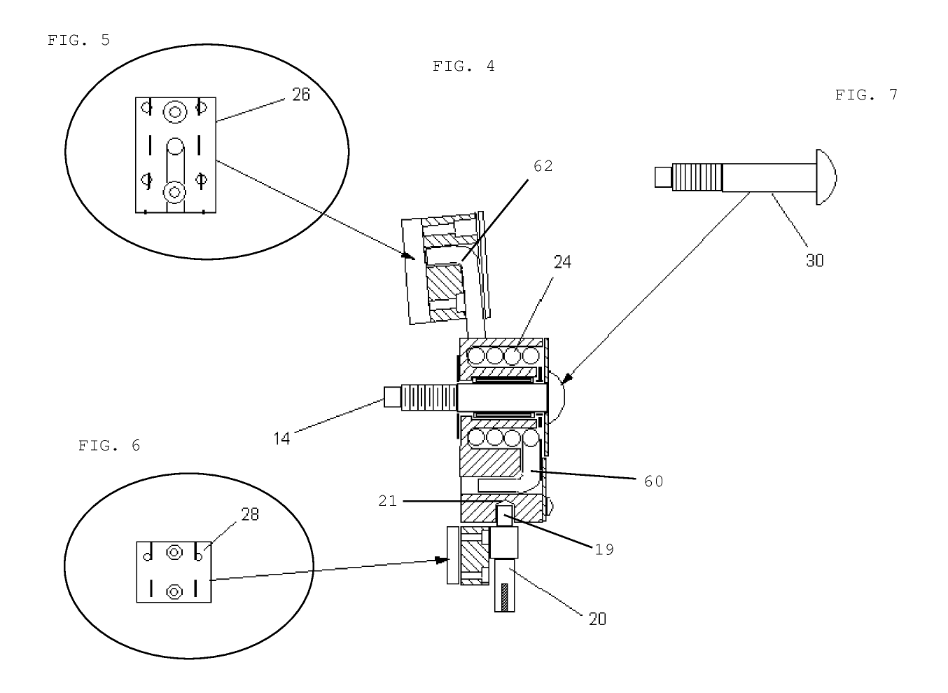

Counterbalance spring 24 (inside pivot 14 and shown on FIG. 4)

Engagement mechanism 20

Thigh cuffs 22 providing a lower body engaging portion

FIGS. 4 to 7 illustrate pivot 14 in greater detail and include a spring 24 mounted within pivot 14. An anchor block 26 is positioned toward a top end of pivot 14 and engages an upper arm 16 while a lower arm 18 engages a main body 32 of pivot 14 via releasable latching anchor 28. A central screw 30 extends through body 32 through spring 24 and also provides a pivot axis for pivot 14. A spring loaded pin 19 may be included at a top of engagement mechanism 20. Pin 19 may be selectively engaged (extended as shown) or disengaged (by retracting or moving downward with lower arm 18 and engagement mechanism 20) from a recess 21 in main body 32 to provide selective engagement or disengagement of the spring assist device of TAO 100.

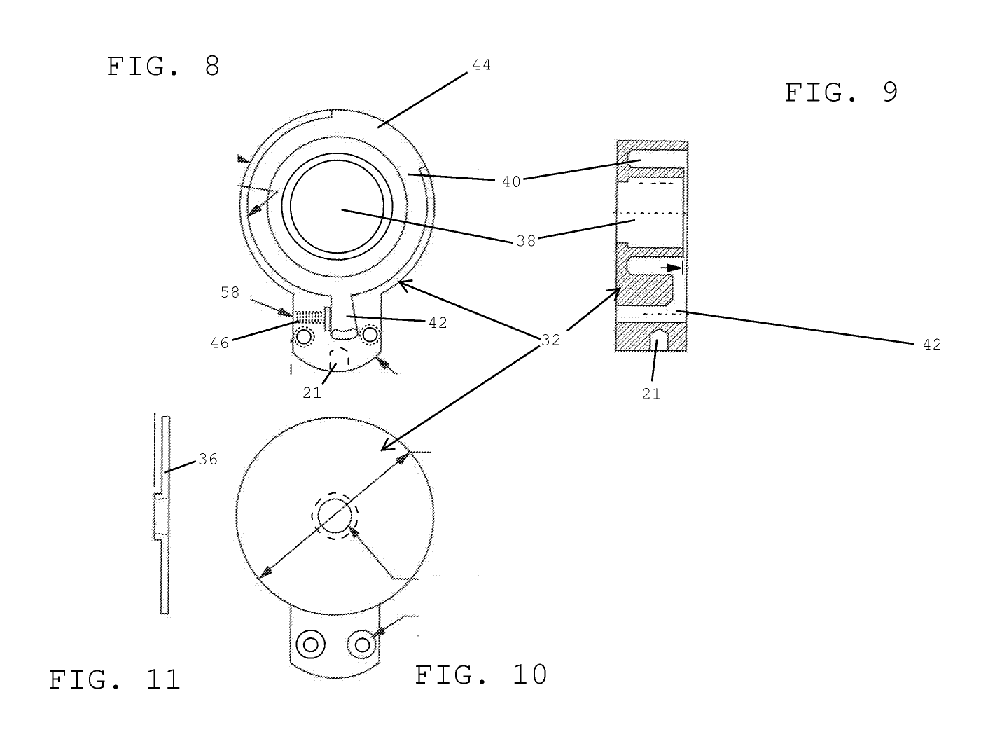

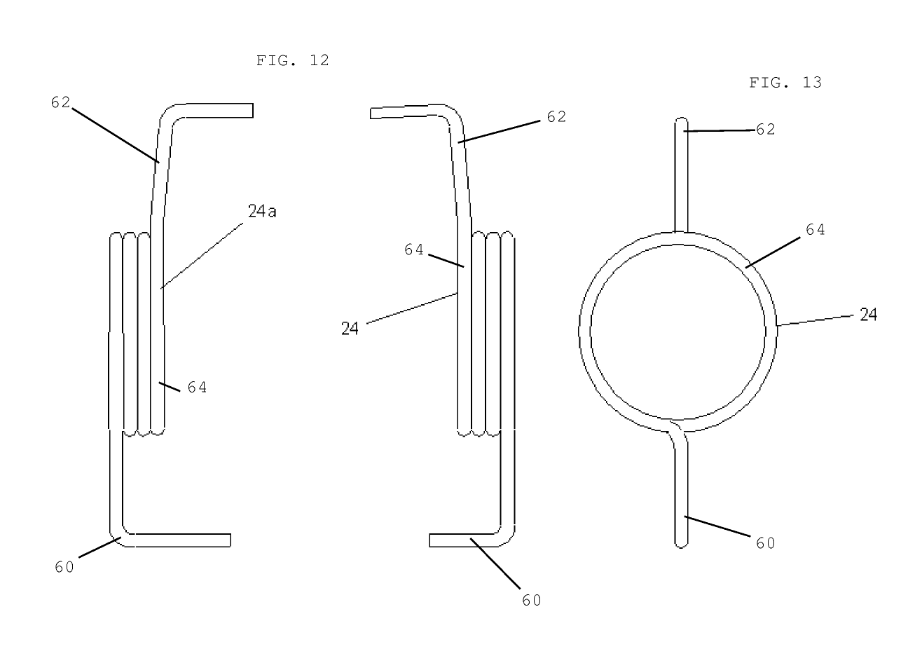

FIGS. 8 to 11 illustrate main body 32 in more detail along with a mating cover 36. Main body 32 may include an opening 38 for receiving screw 30 and possibly some form of a bearing to reduce friction and wear in rotation of the body about the screw. A recess 40 about the screw opening permits the insertion and anchoring of spring 24 within body 32. A lower extension 42 of recess 40 permits a lower arm 60 (as shown in FIG. 4) of spring 24 to be anchored to body 32 while an upper opening 44 in recess 40 permits an upper arm 62 (as shown in FIG. 4) of spring 24 to extend outside body 32 to engage anchor block 26. A preload adjustment screw 58 may be mounted within an opening 46 extending into lower extension 42. Preload screw 58 may press forward against the lower arm of spring 24 and extension 42 may be configured to permit some movement of the lower arm in response to pressure from the preload screw.

Moving the lower arm of spring 24 forward by pressing against it with the preload screw will serve to preload the spring with some tension so that with the latch engaged, there will be immediate resistance to forward movement of upper arm 16. This will serve to provide a quicker response and halting of a forward collapse of the user's trunk.

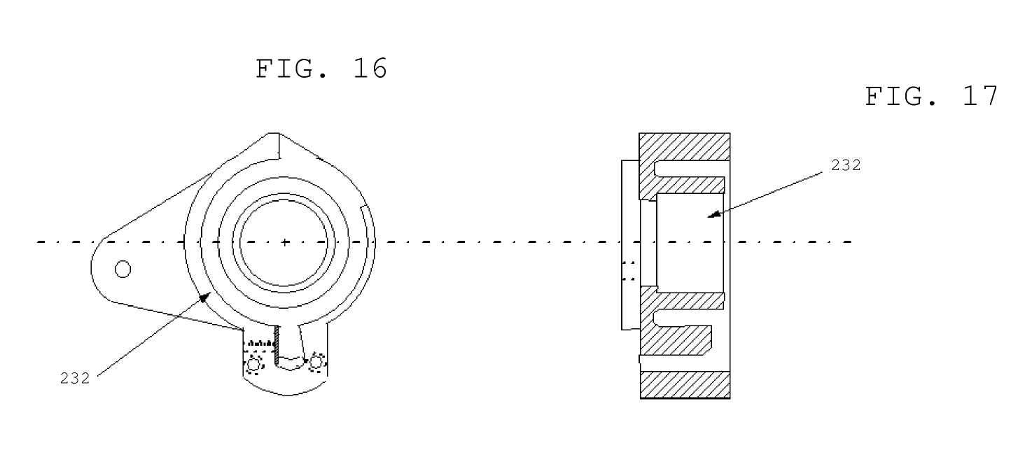

Alternatively, as shown in FIGS. 14 and 15, an alternative main body 132 may be configured without a means for providing a preload to spring 24. With the exception of the preload screw provisions, main bodies 32 and 132 may otherwise be similarly configured.

FIGS. 16 and 17 show another alternative main body 232 which is configured to utilize a conventional latching mechanism such as that incorporated into conventional RGO's and eliminates the need for an external latching pin. Use of main body 232 in a TAO according to the present disclosure may permit a simplified installation and may increase the overall ease of use of the TAO by a patient.

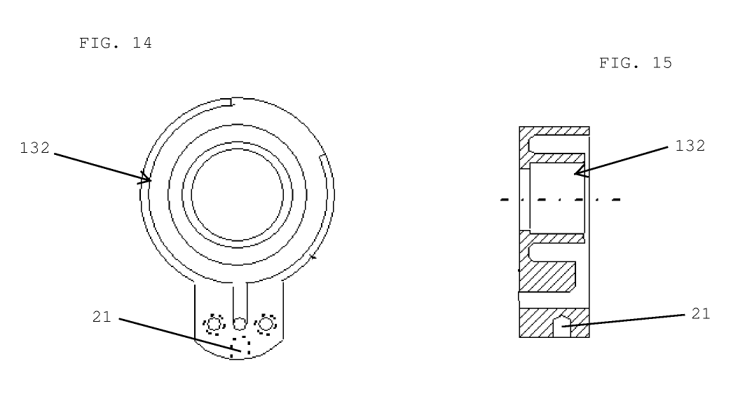

FIG. 12 illustrates spring 24 for use in pivot 14 and a spring 24a for use in a left side or mirror image pivot 14a. These springs are similarly configured with upper arms 62 and lower arms 60 and a central coil 64.

Camptocormia, a syndrome which is increasingly associated with Parkinson's Disease, causes patients to exhibit a severe, bent over posture when walking, standing or sitting. There is currently no recognized medical treatment or orthopedic device to mitigate this degenerative condition, and because of this, the TAO was developed. The TAO is designed to support the torso using a pelvic girdle and chest support while transferring the associated forces through stiff torsion springs to the legs with thigh cuffs. Each of these components should be custom-molded to fit the individual patient but it is anticipated that versions or embodiments with adjustment features may be provided in standard sizes so that TAOs according to the present disclosure may be more readily available and adaptable to users at a variety of price points.

Fitted with a TAO, when a standing patient desires to bend over, all that's required is to lean forward and relax. The individually-sized (or adjusted) torsion springs will preferably match the weight of the patient so that the torso is essentially weightless. Once the task in the bent over position is completed, the patient is free to resume an erect position with only minimal muscle effort. Alternatively, it is anticipated that the assist provided by TAO 100 may be adjusted to match the muscular capabilities of the user. A patient may have some muscular strength to perform some of the lifting of the weight of their own torso and may only require a lesser assistance from TAO 100. Other patients or users may have lost all or substantially all of their muscular ability to raise their trunk and may require TAO 100 to be able to assist in moving substantially all of the weight of their torso to an upright position.

Since the springs assert little resistance near the vertical position, the TAO only minimally impedes normal walking. However, a fully relaxed spring also has no restorative force, so a small amount of spring preload may be desired to ensure that the patient is held fully erect. For this reason, the device incorporates a feature that permits adding varying amounts of preload to the torsion spring with an internal adjustment screw. However, the amount of preload is kept to a minimum to minimize pressure on the thigh cuffs when walking

The current spring mechanism may be selectively engaged and disengaged with a cam operated, spring-loaded indexing pin 19 which may selectively engage or disengage recess 21. Engagement/disengagement of pin 19 from recess 21 may be accomplished with a convenient paddle switch allowing easy access by the user, but other latching mechanisms are possible, including the utilization of existing RGO latching mechanisms. All moving parts may be mated with precision roller bearings to ensure reliability and smooth operation. It is anticipated that other suitable bearings, bushings or similar pivot schemes may be used within the scope of the present disclosure and it is not intended to limit the disclosure to any particular rotational bearing or support arrangement. The pivot mechanism may be fitted with stops and designed to snap into a locked position by simply standing erect.

In order to assume a seated position, such as illustrated in FIG. 3, the spring mechanism must be disengaged. An additional set of stops ensure that the patient cannot fall forward in the seated position.

While TAO 100 as illustrated herein is passive and does not assist the patient in transitioning from a seated to standing position, it is anticipated that a powered version could be developed within the scope of the present disclosure which would perform that function. Such an alternative embodiment might include a secondary means of assisting the user in moving from a seated to a standing position to permit the pivots of the TAO to be re-engaged.

The TAO has shown in patient testing to provide the assistance needed for patients with advanced camptocormia to stand erect, walk normally, bend and reach, and return to a normal standing position. Prior to this, posture was bent over characteristic of the syndrome and mobility was restricted to a walker. Patients in the early stages of camptocormia will also find relief since they are easily fatigued and can only stand erect for limited periods. For those patients, the TAO springs can be sized to provide less assistance and be less intrusive. Over time, the spring sizes and/or preload can be adjusted as needed.

TAO 100 is not intended to be used exclusively to assist camptocormia patients. Others afflicted with an inability to stand erect unassisted may be aided by the device. The main qualifier is an individual's ability to lie on flat surface with the spine straightened and then also be able to stand and bend at the waist without discomfort.

While the invention has been described with reference to preferred embodiments, it is to be understood that the invention is not intended to be limited to the specific embodiments set forth above. Thus, it is recognized that those skilled in the art will appreciate that certain substitutions, alterations, modifications, and omissions may be made without departing from the spirit or intent of the invention. Accordingly, the foregoing description is meant to be exemplary only, the invention is to be taken as including all reasonable equivalents to the subject matter of the invention, and should not limit the scope of the invention set forth in the following claims.

What is claimed is:

1. An orthopedic support device comprising:
an upper body engaging portion;
a lower body engaging portion;
a hinged connection between the upper and lower body engaging portions;
a spring assist device mounted at and engaging the hinged connection.

2. The orthopedic support device of claim 1, further comprising the device configured to be worn by a user, the user requiring assistance in holding the user's torso in a vertical position and returning the user's torso to a vertical position from a forward bent over position, and the spring assist device including a spring selected to provide such assistance based on the weight of the user's torso and the degree of assistance required by the user.

3. The orthopedic device of claim 1, further comprising the spring assist device including a coil spring engaging the hinged connection with a first portion of the spring configured to engage the lower body engaging portion and a second portion of the spring configured to engage the upper body engaging portion.

4. The orthopedic assist device of claim 1, further comprising the device configured to be worn by a user, the user requiring assistance in holding the user's torso in a vertical position and returning the user's torso to a vertical position from a forward bent over position, and further comprising the spring assist device selectively disengageable, such that when the spring assist device is engaged, the spring assist device provides resistance to forward movement of the user's torso, and when the spring assist device is disengaged, the spring assist device provides no resistance to forward movement of the user's torso.

5. The orthopedic assist device of claim 4, further comprising the hinged connection permitting a normal range of motion by the user, such that when the spring assist device is disengaged, the user may sit down without the orthopedic device exerting force to bring the user's torso in line with their lower body.

6. The orthopedic assist device of claim 5, further comprising the spring assist device configured to automatically reengage when a seated user returns to a vertical position.

7. The orthopedic assist device of claim 4, further comprising a preload adjustment device to tension the spring and provide resistance to forward movement of the user's torso when the user is in a vertical position.

8. The orthopedic assist device of claim 4, further comprising the spring assist device including a forward torso movement stop and a rearward torso movement stop to limit the range of movement of the user's torso when the spring assist device is engaged.

9. The orthopedic assist device of claim 4, further comprising the disengagement of the spring assist device includes the disengagement of the spring assist device from the lower body engaging portion.

10. The orthopedic assist device of claim 1, further comprising the upper body engaging portion includes a pelvic girdle.

11. The orthopedic assist device of claim 1, further comprising the lower body engaging portion includes a thigh cuff.

12. The orthopedic assist device of claim 1, further comprising the spring assist device includes a coil spring with a main coiled spring body, an upper arm engaging the upper body engaging portion and a lower arm engaging the lower body engaging portion.

13. The orthopedic assist device of claim 12, further comprising a preload spring extending through a main body of the spring assist device and configured to selectively push the lower arm of the coil spring forward.

14. The orthopedic assist device of claim 1, further comprising a spring loaded pin in the lower body engaging portion and a recess in a main body of the spring assist device, the pin being selectively engagable within the recess, wherein when the pin is engaged within the recess, the spring assist device provides resistance to movement in a first direction between the upper body engaging portion and the lower body engaging portion, and wherein when the pin is disengaged from the recess, the spring assist device provides no resistance to movement in the first direction.
